(19) United States
(12) Patent Application Publication
(10) Pub. No.: US 2016/0206283 A1
(43) Pub. Date: Jul. 21, 2016

(54) ULTRASOUND DIAGNOSTIC APPARATUS

(71) Applicant: KONICA MINOLTA, INC., Tokyo (JP)

(72) Inventor: Kazushi OTA, Tokyo (JP)

(73) Assignee: KONICA MINOLTA, INC., Tokyo (JP)

(21) Appl. No.: 14/989,411

(22) Filed: Jan. 6, 2016

(30) Foreign Application Priority Data
Jan. 21, 2015 (JP) 2015-009059

(51) Int. Cl. A61B 8/00

(52) U.S. Cl. CPC A61B 8/4245; A61B 2576/00

(57) ABSTRACT

An ultrasound diagnostic apparatus, including: an ultrasound probe; a storage which stores an examination point and a direction to locate the ultrasound probe so as to be associated with each other, the examination point being set for a region that is an examination target, and the direction to locate the ultrasound probe being set for the examination point in advance; a display; a display controller; and an inputter, wherein the inputter receives an input operation of specifying the examination point, and the display controller controls the display to display a region mark including a first region figure which shows a shape of the region and a probe mark which is located at a position corresponding to the examination point specified by the input operation and indicates the direction to locate the ultrasound probe.

### BACKGROUND OF THE INVENTION

1. Field of the Invention

The present invention relates to an ultrasound diagnostic apparatus.

2. Description of Related Art

Conventionally, there have been ultrasound diagnostic apparatuses which provide diagnostic information regarding internal configurations of subjects by emitting ultrasound waves from ultrasound probes to the inside of the subjects, receiving the reflected waves, processing the obtained signals to generate ultrasound images reflecting the internal configurations of the subjects, and displaying the generated ultrasound images on display sections. Such ultrasound diagnostic apparatuses are used for human medical care as non-invasive diagnostic apparatuses, for example.

The ultrasound diagnostic apparatus which diagnoses the human body as the subject can display a region mark (body mark) together with the ultrasound image, the region mark showing an examination point where examination was performed in the region as an examination target of the subject. The region mark generally includes a figure which shows the shape of the region in the subject and a probe mark which is displayed at a position corresponding to the examination point in the figure and shows the direction to locate the ultrasound probe. Since such region mark is displayed together with the ultrasound image, the operator of the ultrasound diagnostic apparatus can visually confirm the examination point and the direction to locate the ultrasound probe in the examination easily.

Conventionally, the position and the direction of probe mark have been set based on the input operation by the operator. For example, Japanese Patent Application Laid Open Publication No. 2014-150804 discloses an ultrasound diagnostic apparatus which displays a figure showing the shape of a region in the subject and an angle setting guide for selecting the direction of probe mark on a display screen that is provided with a touch panel. In the ultrasound diagnostic apparatus disclosed by Japanese Patent Application Laid Open Publication No. 2014-150804, the touch panel detects the respective positions where the operator touches the display screen displaying the figure and the angle setting guide, and the position and direction of probe mark in the region mark are set based on the respective detected positions to display the region mark.

However, conventional ultrasound diagnostic apparatuses have a problem that the input operation is complicated since the input operation needs to be performed for both the position and direction of probe mark in the region mark in order to display the region mark. The conventional ultrasound diagnostic apparatuses also have a problem that the operator easily makes mistake in the setting since the operator needs to specify the appropriate position of probe mark from among similar regions and examination points to perform setting.

### SUMMARY OF THE INVENTION

An object of the present invention is to provide an ultrasound diagnostic apparatus which can display a probe mark easily and appropriately.

In order to achieve the above object, according to one aspect of the present invention, there is provided an ultrasound diagnostic apparatus, including: an ultrasound probe which transmits ultrasound waves to a predetermined subject and receives reflected waves of the transmitted ultrasound waves; a storage which stores an examination point and a direction to locate the ultrasound probe so as to be associated with each other, the examination point being set for a region that is an examination target in the subject, and the direction to locate the ultrasound probe being set for the examination point in advance; a display; a display controller which controls display by the display section; and an inputter which receives an input operation from outside, wherein the input inputter receives an input operation of specifying the examination point, and the display controller controls the display to display a region mark including a first region figure which shows a shape of the region and a probe mark which is located at a position corresponding to the examination point specified by the input operation and indicates the direction to locate the ultrasound probe that is set for the examination point.

Preferably, the display controller controls the display to display a plurality of examination point marks showing examination points according to the region, and the input inputter receives an input operation of selecting one of the plurality of examination point marks displayed on the display as the input operation of specifying the examination point.

Preferably, the display controller controls the display to display the plurality of examination point marks by assigning the plurality of examination point marks to positions corresponding to the respective examination points according to the plurality of examination point marks in a second region figure which shows the shape of the region.

Preferably, the ultrasound diagnostic apparatus further includes an image generator which generates an ultrasound image based on the reflected waves received by the ultrasound probe, wherein the display controller controls the display to display the ultrasound image together with the plurality of examination point marks.

Preferably, the display controller controls the display to display an image including the plurality of examination point marks so as to be superposed on a predetermined position of the ultrasound image.

Preferably, the ultrasound diagnostic apparatus further includes an image generator which generates an ultrasound image based on the reflected waves received by the ultrasound probe, and the display controller controls the display to display the ultrasound image together with the region mark after the display displays the region mark.

Preferably, the inputter includes a touch panel which is disposed so as to be superposed on a display screen of the display, and the inputter receives the input operation by detecting touch to a surface of the touch panel.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a view showing a schematic configuration of an ultrasound diagnostic apparatus in an embodiment of the present invention;

FIG. 2 is a block diagram showing a main functional configuration of the ultrasound diagnostic apparatus;

FIG. 3 is a view for explaining an example of an examination region selection screen;

FIG. 4 is a view for explaining an example of an ultrasound diagnostic screen;

FIG. 5 is a view for explaining an example of the ultrasound diagnostic screen including an examination point selection image;

FIG. 6 is a view showing an enlarged example of the examination point selection image;

FIG. 7 is a view for explaining an example of the ultrasound diagnostic screen displaying a body mark;

FIG. 8 is a flowchart showing a control procedure of image file generation processing;

FIG. 9 is a flowchart showing a control procedure of body mark display processing; and

FIG. 10 is a flow chart showing a control procedure of image file generation processing according to a modification example.

### DETAILED DESCRIPTION OF THE PREFERRED EMBODIMENT

Hereinafter, an embodiment according to the ultrasound diagnostic apparatus of the present invention will be described with reference to the drawings.

FIG. 1 is a view showing the schematic configuration of the ultrasound diagnostic apparatus 1 in the embodiment of the present invention.

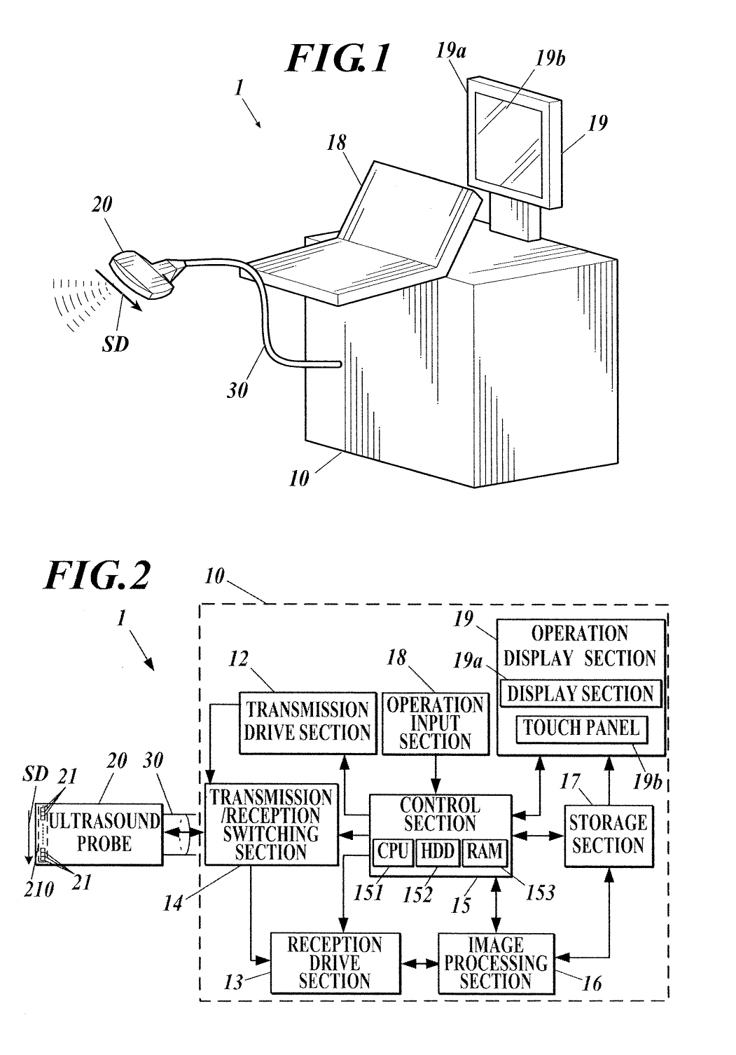

FIG. 2 is a block diagram showing the main functional configuration of the ultrasound diagnostic apparatus 1.

As shown in FIG. 1, the ultrasound diagnostic apparatus 1 includes an ultrasound diagnostic apparatus main body 10 and an ultrasound probe 20 which is connected to the ultrasound diagnostic apparatus main body 10 via a cable 30. The ultrasound diagnostic apparatus main body 10 is provided with an operation, input section 18 and an operation display section 19 which includes a display section 19a (display) and a touch panel 19b (inputter). On the basis of an input operation to an input device such as a keyboard and a mouse of the operation input section 18 by the operator and a touch operation to the touch panel 19b of the operation display section 19 by the operator, a control section 15 of the ultrasound diagnostic apparatus main body 10 outputs drive signals to the ultrasound probe 20 to output ultrasound waves, performs various types of processing by obtaining reception signals according to the reception of ultrasound waves from the ultrasound probe 20, and displays results and such like on the display section 19a as needed.

As shown in FIG. 2, the ultrasound diagnostic apparatus main body 10 includes a transmission drive section 12, a reception drive section 13, a transmission reception switching′ section 14, the control section 15 (display controller), an image processing section 16 (image generator), a storage section 17, an operation input section 18, an operation display section 19 and such like.

The transmission drive section 12 outputs a pulse signal (drive signal) to be supplied to the ultrasound probe 20 in accordance with a control signal input from the control section 15, and makes the ultrasound probe 20 generate ultrasound waves. The transmission drive section 12 includes, for example, a clock generation circuit, a pulse generation circuit, a pulse width setting section and a delay circuit. The clock generation circuit is a circuit which generates a clock signal for determining the transmission timing and transmission frequency of pulse signal. The pulse generation circuit is a circuit which generates bipolar type square-wave pulses having a preset voltage amplitude at a predetermined cycle. The pulse width setting section sets the pulse width of square-wave pulses output from the pulse generation circuit. The square-wave pulses generated in the pulse generation circuit are separated to different connection paths for respective individual oscillators 21 of the ultrasound probe 20 before or after the input to the pulse width setting section. The delay circuit is a circuit which delays the outputs by respective delay periods which are respectively set for the connection paths.

The reception drive section 13 is a circuit which obtains a reception signal input from the ultrasound probe 20 in accordance with the control of control section 15. The reception drive section 13 includes, for example, an amplifier, an A/D conversion circuit and a phasing addition circuit. The amplifier amplifies reception signals at a predetermined amplification factor which is set in advance, the reception signals corresponding to ultrasound waves received by respective oscillators of the ultrasound probe 20. The A/D conversion circuit converts the amplified reception signals to digital data at a predetermined sampling frequency. With respect to the reception signals which were A/D converted, the phasing addition circuit adjusts the time phase by providing delay periods for respective connection paths corresponding to the oscillators, and performs addition thereof (phasing addition) to generate sound ray data.

The transmission reception switching section 14 performs a switching operation on the basis of control of the control section 15 so as to make the transmission drive section 12 transmit drive signals to the oscillators 21 when the oscillators 21 oscillate ultrasound waves, and to make the reception drive section 13 output reception signals when the obtaining signals according to the emitted ultrasound waves.

The control section 15 includes a CPU 151 (Central Processing Unit), an HDD 152 (Hard Disk Drive) (storage), a RAM 153 (Random Access Memory) and such like. The CPU 151 reads out various programs stored in the HDD 152, loads the read programs into the RAM 153, and integrally controls the operations of sections in the ultrasound diagnostic apparatus 1 in accordance with the loaded programs. The HDD 152 stores control programs for operating the ultrasound diagnostic apparatus 1, various processing programs, various types of setting data, image files generated in the ultrasound diagnostic apparatus 1 and such like. The setting data includes data in which an examination point set for each region of the subject is associated with the direction (probe direction) to locate the ultrasound probe 20, the direction being set in advance according to the examination point, and predetermined examination point image data. The examination point, probe direction and examination point image will be described in detail later. These programs and setting data may be stored so as to be writable/readable and updatable in an auxiliary storage device using a non-volatile memory such as a flash memory in addition to the HDD 152, for example. The RAM 153 is a volatile memory such as a SRAM and a DRAM, and provides a working memory space to the CPU 151 to store temporal data.

Separately from the CPU 151 of the control section 15, the image processing section 16 performs arithmetic processing for generating an ultrasound image (diagnostic image) based on the reception data of ultrasound waves. The ultrasound image may include image data which is displayed on the operation display section 19 nearly real time, moving image data formed of a series of image data, still image data of snapshot and such like. The arithmetic processing may be performed by the CPU 151.

The storage section 17 is a volatile memory such as a DRAM (Dynamic Random Access Memory), for example. Alternatively, the storage section 17 may be various non-volatile memories which can rewrite rapidly. The storage section 17 stores, for each frame, image data of ultrasound images for real time display processed in the image processing section 16. The image data stored in the storage section 17 is read out in accordance with the control of control section 15 to be transmitted to the display section 19a or output outside the ultrasound diagnostic apparatus 1 via a communication section not shown in the drawings. At this time, when the display type of display section 19a is television system, a DSC (Digital Signal Converter) may be provided between the storage section 17 and the display section 19a to perform the output after converting the scanning format.

The operation input section 18, which includes a push button switch, keyboard, mouse or trackball, or combinations thereof, converts the input operation by the operator to an operation signal and outputs the signal to the control section 15.

The display section 19a of the operation display section 19 includes a display screen and a drive section thereof, the display screen using any one of various display systems such as an LCD (Liquid Crystal Display), an organic EL (Electro-Luminescence) display, an inorganic EL display, plasma display, and a CRT (Cathode Ray Tube) display. The display section 19a generates a drive signal of display screen. (each display pixel) in accordance with the control signal output from the CPU 151 and the image data generated in the image processing section 16. The display section 19a then displays menus according to ultrasound diagnosis, status, measurement data such as ultrasound images based on the received ultrasound waves and the like on the display screen.

The touch panel 19b of the operation display section 19 is a capacitance type touch panel which is disposed so as to be superposed on the display screen of the display section 19a. The touch panel 19b detects a touch operation on the basis of the change in capacitance between the internal conducting layer and the surface caused by a fingertip or the like of operator touching the surface, and outputs a signal indicating the detected position as an operation signal to the control section 15. The type of touch panel 19b is not limited to the capacitance type, and may be other types such as resistive type and electromagnetic induction type.

The operation input section 18 and the operation display section 19 may be integrally provided to the housing of ultrasound diagnostic apparatus main body 10, or may be attached to the outside via a USB cable and such like. When the ultrasound diagnostic apparatus main body 10 includes an operation input terminal or a display output terminal, conventional peripheral equipment for operation and display may be connected to the terminal to be used.

The ultrasound probe 20 functions as a sound sensor which oscillates ultrasound waves (here, approximately 1 to 30 MHz) to transmit (emit) the ultrasound waves to a subject such as a living body, receives reflected waves (echo) reflected from the subject among the transmitted ultrasound waves and converts the reflected waves into electric signals. The ultrasound probe 20 includes an oscillator array 210 which is an array of a plurality of oscillators 21 which transmit and receive the ultrasound waves.

The oscillator array 210 is an array of a plurality of oscillators 21 which include piezoelectric elements having piezoelectric bodies and electrodes provided at the both ends of each of the piezoelectric bodies generating charges by deformation (expansion and contraction) of the piezoelectric bodies. The piezoelectric bodies are deformed according to the electric field generated at the respective piezoelectric bodies by the supply of voltage pulse (pulse signal) to the oscillators 21, and the ultrasound waves are transmitted. When ultrasound waves in a predetermined frequency band are emitted to the oscillators 21, the sound pressure changes (vibrates) the thickness of piezoelectric bodies, thereby the electric field corresponding to the change amount is generated at both ends in the thickness change direction of the piezoelectric bodies, and charge in the amount corresponding to the charge is induced at the electrodes on both ends of piezoelectric element. Here, a ferroelectric is used as a piezoelectric body.

As for the ultrasound probe 20 in the embodiment, the oscillator array 210 includes 192 oscillators 21 in one-dimensional array in a predetermined oscillator array direction. The oscillators 21 may also be arrayed in a direction orthogonal to the oscillator array direction to be two-dimensionally arrayed. The number of oscillators 21 may be set arbitrarily. The ultrasound probe 20 in the embodiment transmits ultrasound waves from a set of successive oscillators 21 (for example, 64 oscillators 21) among the 192 oscillators 21 on the basis of pulse signals from the transmission drive section 12. Every time the ultrasound waves are generated, the set of oscillators 21 transmitting ultrasound waves is shifted by the predetermined number of oscillators 21 in the oscillator array direction, and thereby scanning is performed in the scanning direction SD parallel to the oscillator array direction. The ultrasound probe 20 used in the embodiment is a linear electronic scanning type in which the transmission directions of respective ultrasound waves transmitted at different timings are parallel to each other. The ultrasound probe 20 may adopt any one of various electronic scanning types such as sector electronic scanning type and convex electronic scanning type and various mechanical scanning types such as linear scanning type, sector scanning type, arc scan type and radial scan type. The band width of reception frequency of ultrasound waves in the ultrasound probe 20 may be set arbitrarily.

The ultrasound diagnostic apparatus 1 may be configured so that any one of a plurality of different ultrasound probes 20 is connected to the ultrasound diagnostic apparatus main body 10 to be used according to the diagnosis target.

The cable 30 has, at one end thereof, a connector (not shown in the drawings) to the ultrasound diagnostic main body 10, and the ultrasound probe 20 is configured so as to be attachable to and detachable from the ultrasound diagnostic apparatus main body 10 via the cable 30.

Next, the display operation of body mark (region mark) executed by the ultrasound diagnostic apparatus 1 in the embodiment will be described.

The ultrasound diagnostic apparatus 1 emits ultrasound waves from the ultrasound probe 20 to an examination point of a region (for example, a hand or a leg) which is an examination target in a human body as a subject, receives the reflected waves, and on the basis of the obtained reception signals, displays an ultrasound image reflecting the internal configuration of the subject on the display section 19a. The ultrasound diagnostic apparatus 1 displays a body mark together with the ultrasound image, the body mark including a figure which shows the shape of the region in the subject and a probe mark which is located at a position corresponding to the examination point in the figure and shows the direction (probe direction) to locate the ultrasound probe 20 at the examination point. In the embodiment, the probe direction is a direction parallel to the scanning direction SD. Image data of an image obtained by combining the body mark and the ultrasound image is stored in the storage section 17 or the HDD 152. Since both of the ultrasound image and the body mark are displayed on the display section 19a, the operator of the ultrasound diagnostic apparatus 1 can easily visually confirm the examined region in the subject which is shown by the ultrasound image, and the examination point and direction of ultrasound probe 20 located at the examination point in the region of subject to generate the ultrasound image.

In general examinations and health diagnoses using the ultrasound diagnostic apparatus 1, there are performed, in many cases, typical examinations (routine examinations) in which the direction to locate the ultrasound probe 20 is determined in advance for each examination point in the examination region. The embodiment will be described by using an example in which the typical examination is performed by the ultrasound diagnostic apparatus 1.

In a case where a lesion is found by the typical examination, for example, an examination (non-typical examination) may be performed by locating the ultrasound probe 20 in a direction different from the predetermined direction in addition to the typical examination. When the direction of probe mark is to be set in the non-typical examination, the processing for directly setting the direction of probe mark is performed by an input operation from the operator, for example, instead of the method shown in the embodiment.

Hereinafter, the body mark display operation will be described by using an example of examination regarding a joint of hand (finger) by the ultrasound diagnostic apparatus 1.

When the examination by the ultrasound diagnostic apparatus 1 is started, an examination region selection screen D1 for making the operator select the examination region is displayed on the display section 19a.

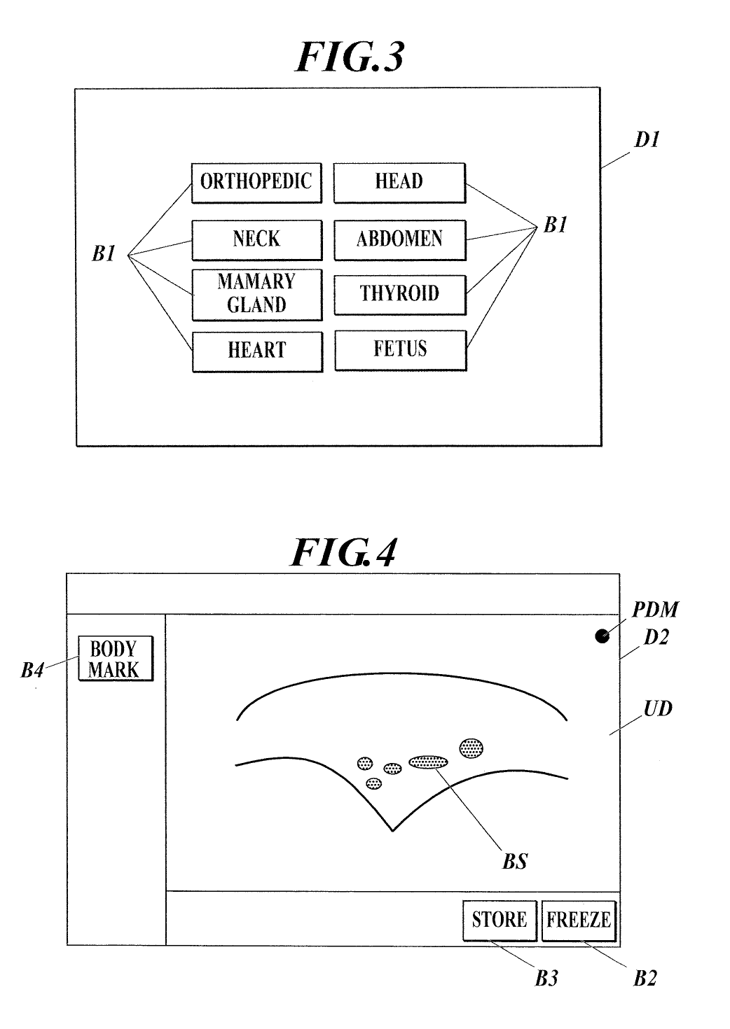

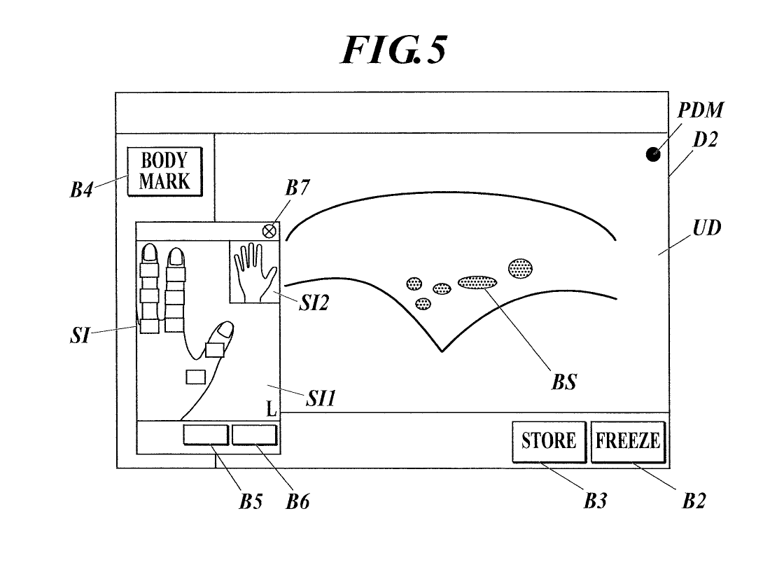

FIG. 3 is a view for explaining an example of the examination region selection screen D1. The examination region selection screen D1 displays a plurality of examination region selection buttons B1 showing candidates of the region (examination region) to perform examination such as orthopedic, (finger, elbow, shoulder, knee, ankle and toe), head, neck, abdomen, mammary gland, thyroid, heart and fetus. The examination region is selected by a touching operation to the touch panel 19b at a position corresponding to one of the plurality of examination region selection buttons B1. When the touch operation to the touch panel 19b is performed at the position corresponding to the “orthopedic”, the display of examination region selection button B1 is changed to the display showing candidates of examination region included in the “orthopedic” category such as finger, elbow, shoulder, knee, ankle and toe. Then, the examination region is selected by a touch operation to the touch panel 19b at a position corresponding to one of the examination region selection buttons B1. When the examination region is selected, the transmission of ultrasound waves to subject and the reception of the reflected waves are started, and an ultrasound diagnostic screen including an ultrasound image is displayed on the display section 19a on the basis of the received reflected waves.

FIG. 4 is a view for explaining an example of ultrasound diagnostic screen D2. The ultrasound diagnostic screen D2 includes an ultrasound image UD, a probe direction mark PDM, a freeze button B2, a storage button B3 and a body mark input mode shift button B4. The probe direction mark PDM is a circular mark for showing the scanning direction of ultrasound waves transmitted from the ultrasound probe 20. In the embodiment, the probe direction mark PDM indicates that the ultrasound scanning was performed from an end portion (right end in FIG. 4) where the probe direction mark PDM is provided in the ultrasound image UD toward an end portion (left end in FIG. 4) where the probe direction mark PDM is not provided. The freeze button B2 is touched when the operator is to stop the ultrasound image UD, which is being updated and displayed (movie display) nearly real time at each transmission and reception of ultrasound waves, to switch the display to fixed display (still image display). The freeze button B2 is also touched when the operator is to switch the still image display to the movie display. The storage button B3 is touched when the ultrasound image UD is to be stored in the HDD 152. The body mark input mode shift button B4 is touched when the operator is to execute the body mark display processing for performing display by adding a body mark to the ultrasound image UD.

The ultrasound image UD shown in FIG. 4 is an image based on data measured in a Doppler mode by a color Doppler method regarding a finger of a hand. In FIG. 4, blood signals BS showing the blood stream generated in a joint synovium of a finger of a hand is displayed so as to be colored.

In the ultrasound diagnostic apparatus 1, when a touch operation to the touch panel 19b is performed at a position corresponding to the body mark input mode shift button B4 on the ultrasound diagnostic screen D2, the operation of body mark input mode is performed. In the body mark input mode, the examination point selection image SI is displayed so as to be superposed (pop-up display) on a part of the ultrasound diagnostic screen D2.

FIG. 5 is a view for explaining an example of the ultrasound diagnostic screen D2 including the examination point selection image SI.

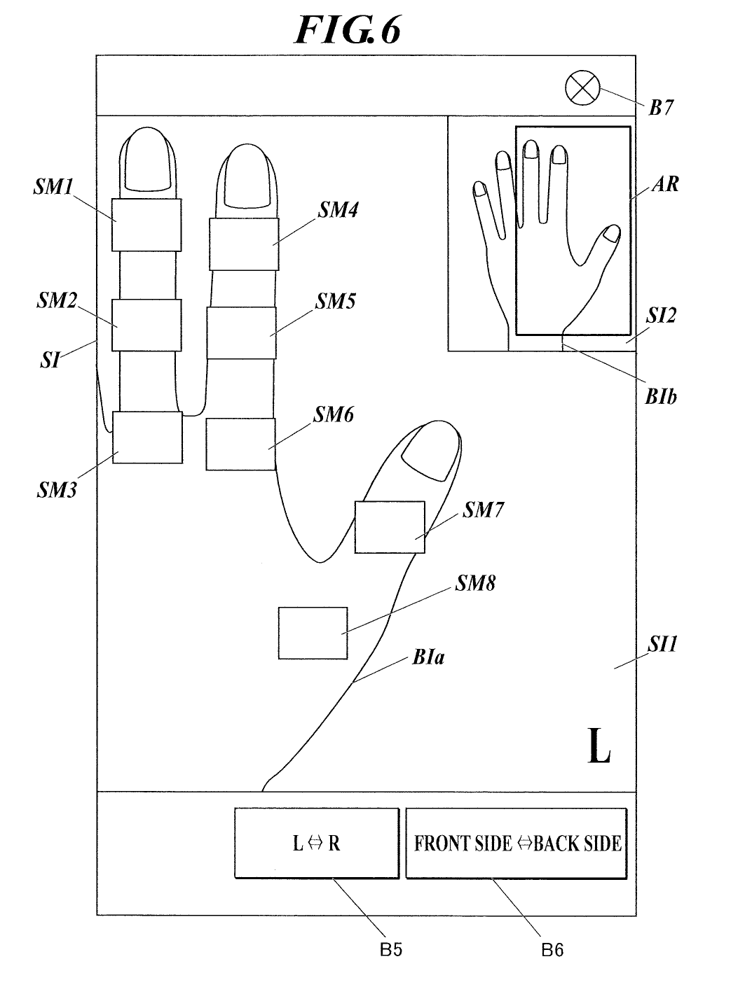

FIG. 6 is a view showing an enlarged example of the ultrasound point selection image SI.

The examination point selection image SI is an image having a rectangular area which is pop-up displayed near the left end of the ultrasound diagnostic screen D2. The examination point selection image SI is displayed so as to overlap a part of the ultrasound image UD at a predetermined position of the ultrasound image UD. Preferably, the examination point selection image SI is displayed so as to overlap a position near an end part excluding the main part of the ultrasound image UD; however, the examination point selection image SI may be displayed so as to overlap a position including the main part. The examination point selection image SI may also be displayed at a position not overlapping the ultrasound image UD in the ultrasound diagnostic screen D2. The examination selection image SI includes a main image SI1, preview image SI2, a left and right switching button B5, a front back switching button B6 and a close button B7.

In the main image SI1, a region figure BIa (second region figure) is displayed, the region figure BIa showing the thumb, index finger and middle finger of the hand which is selected as the examination region and seen from the back side of the hand. In the main image SI1, examination point marks SM1 to SM8 (examination point marks) (hereinafter, merely described as examination point marks SM) are displayed, the examination point marks being formed of rectangular marks which show a plurality of examination points that are set for the hand as the examination region. The examination point marks SM are assigned to positions corresponding to respective examination points according to the examination point marks SM in the region figure BIa, and displayed so as to overlap the region figure BIa. Here, as the examination points set for the hand, there are IP joint (interphalangeal joint) and MP joint (metacarpophalangeal joint) of the thumb, and DIP joint (distal interphalangeal joint), PIP joint (proximal interphalangeal joint) and MP joint of each of the index finger, middle finger, ring finger and little finger, for example. The examination point marks SM1 to SM8 shown in the main image SI1 of FIG. 6 respectively show the examination points set at left middle finger DIP joint, left middle finger PIP joint, left middle finger MP joint, left index finger DIP joint, left index finger PIP joint, left index finger MP joint, left thumb IP joint and left thumb MP joint. The examination point marks are displayed at positions corresponding to the respective examination points. The letter “L” indicating left hand is displayed near the lower right end of the main image SI1.

By a predetermined touch operation being performed to the touch panel 19b in the main image SI1, the region figure BIa included in the main image SI1 and elements including the examination point marks SM are enlarged, reduced or moved in parallel (panning). Specifically, when two points in the main image SI1 are touched on the touch panel 19b and the movement of touched positions are detected in a direction increasing the distance between the touched two points, the elements included in the main image SI1 are displayed to be enlarged according to the increase in the above distance. When two points in the main image SI1 are touched on the touch panel 19b and the movement of touched positions is detected in a direction decreasing the distance between the touched positions, the elements included in the main image SI1 are displayed to be reduced according to the decrease in the distance. When a point in the main image SI1 is touched on the touch panel 19b and the movement of the touched position is detected, the region figure BIa and examination point marks SM are moved in parallel and displayed according to the movement, and the portion of the left hand region figure BIa to be displayed in the main image SI1 is changed.

The preview image SI2 includes a region figure BIb showing the entire left hand and a preview display frame AR showing the range of the region figure BIb corresponding to the region figure BIa displayed in the main image SI1. When a point corresponding to a position in the frame of preview display frame AR is touched on the touch panel 19b and movement of the touched position is detected, in accordance with the movement, the display position of preview display frame AR is moved in the preview image SI2, and the main image SI1 is changed to an image of the portion corresponding to the preview display frame AR in the left hand.

The main image SI1 and the preview image SI2 are displayed on the basis of data stored in the HDD 152 of the control section 15.

The HDD 152 stores examination point image data having a plurality of examination point marks SM provided to respective positions corresponding to a plurality of examination points which are set for a region in the figure data showing the shape of the region of the subject. Examination point image data for one region seen from a plurality of viewpoints is stored as needed. For example, as for the hand as an examination region, the examination point image data according to the examination point of front side and back side is stored in the HDD 152 for each of the left hand and right hand. The main image SI1 and preview image SI2 are displayed by using examination point image data according to the region selected on the examination region selection screen D1 in FIG. 3 from among examination point image data according to regions stored in the HDD 152.

The left and right switching button B5 is a button which is touched when the operator is to switch the display content of the main image SI1 and preview image SI2 from left hand to right hand, or from right hand to left hand.

The front back switching button B6 is a button which is touched when the operator is to switch the display content of the main image SI1 and preview image SI2 between the front side and back side (for example, between palm side and back side of hand).

The close button B7 is a button which is touched when the operator is to close the examination point selection image SI to end the body mark input mode.

In the examination point selection image SI, when a touch operation is performed to the touch panel 19b at a position corresponding to one of the plurality of examination point marks SM showing examination point candidates to select an examination point mark SM, and the examination point is specified, a body mark BM including a region figure BIc (first region figure) and a probe mark PM is displayed so as to be superposed on the ultrasound image UD.

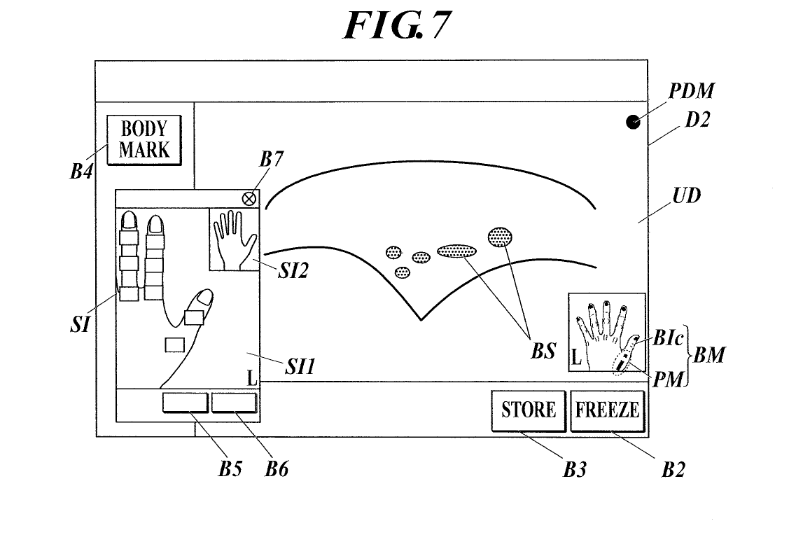

FIG. 7 is a view for explaining an example of ultrasound diagnostic screen D2 displaying the body mark BM. Here, the explanation is made by taking, as an example, a case where the examination point mark SM8 according to the left thumb MP joint is selected in the examination point selection image SI to specify the left thumb MP joint as the examination point.

In the ultrasound diagnostic screen D2 of FIG. 7, the body mark BM having a probe mark PM is displayed so as to be superposed at the position of left thumb MP joint corresponding to the examination point mark SM8 near the lower right end of the ultrasound image UD. The probe mark PM is an “i” shaped mark including an elongate rectangular part and a square part which is located near the rectangular part on the extended line of the rectangular part. The direction of rectangular part of probe mark PM indicates the scanning direction SD of the ultrasound probe 20. The probe mark PM indicates that the scanning direction SD of ultrasound waves is the direction from the square part side toward the rectangular part side. The letter “L” indicating left hand is displayed near the lower left of body mark BM.

Here, the probe mark PM in the body mark BM is displayed so that the direction thereof is consistent with the probe direction of ultrasound probe 20 which is set for the examination point. The HDD 152 of the control section 15 stores a plurality of examination points and probe directions so as to be associated with each other, the plurality of examination points being set for each region of the subject, and the probe directions being set in advance according to the plurality of examination points. When one examination point mark SM in the examination point selection image SI is selected, display is performed by setting the direction of probe mark PM on the basis of the probe direction set for the examination point according to the selected examination point mark SM.

As for the hand as an examination region, the probe direction of ultrasound probe 20 is set to be a direction parallel to the finger direction of each examination point corresponding to the finger joint. Therefore, in the example of FIG. 7, the body mark BM having a probe mark PM parallel to the thumb direction is displayed.

In such way, in the ultrasound diagnostic apparatus 1 in the embodiment, the probe direction of ultrasound probe 20 is set for the examination point of each region of the subject. By selecting an examination point mark SM to specify the examination point in the examination point selection image SI, the body mark BM including the probe mark PM reflecting the probe direction is displayed on the display section 19a.

Next, image file generation processing and body mar display processing included in the image file generation processing will be described, the image file generation processing generating an image file according to the ultrasound image Un which is combined with the body mark BM.

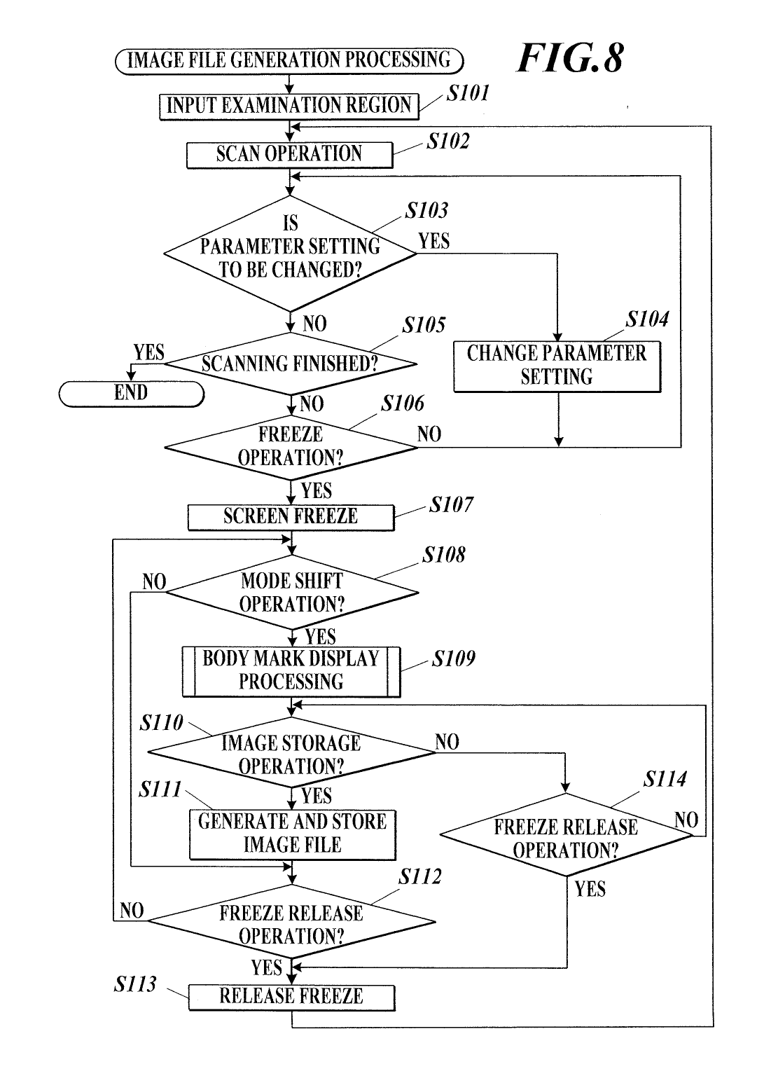

FIG. 8 is a flow chart showing a control procedure of the image file generation processing.

The image file generation processing is processing executed when performing an examination according to a predetermined examination region by the ultrasound diagnostic apparatus 1.

When the image file generation processing is started, the control section 15 executes processing according to the input of examination region (step S101). Here, the control section 15 controls the display section 19a to display the examination region selection screen D1 shown in FIG. 3. When a touch operation to the touch panel 19b is detected at a position corresponding to one of the plurality of examination region selection buttons B1 included in the examination region selection screen D1, the control section 15 performs various types of setting corresponding to the examination region selection button B1 for which the touch operation was detected. Specifically, according to the examination region selection button B1 for which the touch is detected, the control section 15 performs setting of scan operation of step S102 to be described later and setting of examination point selection image SI displayed in step S201 of body mark display processing, and the control section 15 stores the setting in the RAM 153.

Before the processing of step S101, predetermined input processing of patient information for inputting patient information and such like may be performed.

When the processing of step S101 is finished, the control section 15 starts scan operation (step S102). The scan operation is the operation in the ultrasound diagnostic apparatus 1 of generating image data according to the ultrasound image UD by transmitting and receiving ultrasound waves with the ultrasound probe 20 and making the display section 19a display the ultrasound image UD. Specifically, the control section 15 outputs pulse signals from the transmission drive section 12 to the ultrasound probe 20 to make the ultrasound probe 20 perform scanning and transmission of ultrasound waves and make the reception drive section 13 receive reception signals according to reflected waves received by the ultrasound probe 20. The control section 15 controls the image processing section 16 to generate image data according to ultrasound image UD on the basis of the reception signals. Then, the control section 15 controls the storage section 17 to store the generated image data for each frame. On the basis of the image data stored in the storage section 17, the control section 15 controls the display section 19a to display the ultrasound diagnostic screen D2 displaying the ultrasound image UD as shown in FIG. 4, for example.

Here, the control section 15 performs the scan operation at a setting corresponding to the examination region selected in step S101. The setting of scan operation includes a measurement mode, a gain in the processing of reception signals and parameters such as dynamic range, for example. As the measurement mode selected here, there are B mode, A mode and Doppler mode, for example. In the embodiment, since the hand is selected as the examination region, the measurement mode is set to be the Doppler mode by the color Doppler method, and the scan operation is performed in the Doppler mode.

The control section 15 determines whether the operation input section 18 or the operation display section 19 performs input operation according to the change of setting for various parameters (step S103). The various parameters include the gain and dynamic range, for example.

If the control section 15 determines that the setting change operation of parameter was performed (step S103; YES), the control section 15 performs the setting change of parameter corresponding to the input operation to the operation input section 18 or the operation display section 19 (step S104). When the processing of step S104 is finished, the processing proceeds to step S103.

In step S103, if it is not determined that the change operation of parameter setting was performed (step S103; NO), the control section 15 determines whether input operation instructing the end of scan operation was performed to the operation input section 18 (step S105). If it is determined that the input operation instructing the end of scan operation was performed (step S105; YES), the control section 15 ends the image file generation processing.

If it is not determined that the input operation instructing the end of scan operation was performed (step S105; NO), the control section 15 determines whether the freeze operation instructing switching the ultrasound image UD on movie display to the still image display was performed to the operation display section 19 (step S106). The control section 15 determines that the freeze operation was performed when touch to the touch panel 19b is detected at a position corresponding to the freeze button B2 shown in FIG. 4. If it is not determined that the freeze operation was performed (step S106; NO), the processing proceeds to step S103.

If it is determined that the freeze operation was performed (step S106; YES), the control section 15 performs image freeze control to fix the ultrasound image UD and perform still image display to the ultrasound image UD which is displayed on the display section 19a when the freeze operation is performed. The control section 15 also stops the scan operation (step S107).

The control section 15 determines whether the operation display section 19 received the mode shift operation instructing the shift to body mark input mode (step S108). The control section 15 determines that the mode shift operation was performed when touch to the touch panel 19b is detected at a position corresponding to the body mark input mode shift button B4 shown in FIG. 4.

If it is determined that the mode shift operation was performed (step S108; YES), the control section 15 executes after-mentioned body mark display processing (step S109). In the body mark display processing, composite image data combining the ultrasound image UD with the body mark BM is generated, and on the basis of the composite image data, the display section 19a displays an image in which the body mark BM is superposed on the ultrasound image UD as shown, in FIG. 7.

When the body mark display processing in step S109 is finished, the control section 15 determines whether the operation display section 19 received an image storage operation instructing storage of the image (step S110). The control section 15 determines that the image storage operation was performed when touch to the touch panel 19b was detected at a position corresponding to the storage button B3 shown in FIG. 7.

If it is determined that the image storage operation was performed (step S110; YES), the control section 15 generates an image file for storage on the basis of image data according to the image including the ultrasound image UD and the body mark BM displayed in step S109, and the control section 15 stores the image file in the HDD 152 (step S111). Specifically, the control section 15 generates storage image data by converting the composite image data combining the ultrasound image UD with the body mark BM into a predetermined file format. The storage image data after the conversion is a bit map image, for example. The storage image data after conversion may be compressed data which is compressed by a compression method such as JPEG. In a case where the body mark BM is not displayed in the body mark display processing of step S109 (that is, when the processing proceeds from step S206 to step S111 in the after-mentioned body mark display processing), the storage image data is generated from image data according to ultrasound image UD which is not combined with the body mark BM. Next, the control section 15 adds predetermined accompanying information to the generated storage image data to generate an image file formed of image data based on DICOM (Digital Imaging and Communication in Medicine) standard, for example. The control section 15 stores the generated image file in the HDD 152.

The control section 15 determines whether a freeze release operation instructing release of the still image display of ultrasound image UD and shift to the movie display was performed to the operation display section 19 (step S112). The control section 15 determines that the freeze release operation was performed when touch to the touch panel 19b was detected at a position corresponding to the freeze button B2 shown in FIG. 7. If it is not determined that the freeze release operation was performed (step S112; NO), the processing proceeds to step S108. If it is not determined that the mode shift operation was performed in step S108 (step S108; NO), the processing proceeds to step S112.

If it is determined that the freeze release operation was performed (step S112; YES), the control section 15 performs freeze release control for switching the display of ultrasound image UD on the display section 19a from still image display to the movie display (step S113). When the processing of step S113 is finished, the processing proceeds to the processing of step S102.

If it is not determined that the image storage operation was performed in step S110 (step S110; NO), the control section 15 determines whether the freeze release operation was performed (step S114). If it is not determined that the freeze release operation was performed (step S114; NO), the processing proceeds to the processing of step S110. If it is determined that the freeze release operation was performed (step S114; YES), the processing proceeds to the processing of step S113.

The control section 15 performs the above image file generation processing, and ends the image file generation processing when it is determined that the input operation instructing the end of scan operation was performed in step S105.

Next, the body mark display processing executed in step S109 of image file generation processing will be described.

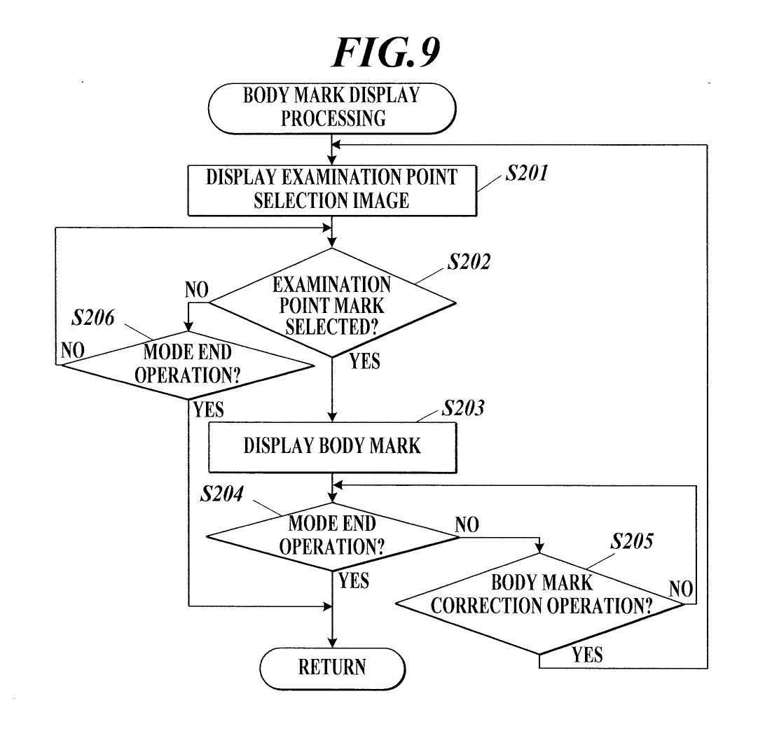

FIG. 9 is a flow chart showing a control procedure of the body mark display processing.

When the body mark display processing is started, the control section 15 displays the examination point selection image SI shown in FIGS. 5 and 6 so as to be superposed on the ultrasound diagnostic screen D2 (step S201). Specifically, the control section 15 reads out, from the HDD 152, the examination point image data according to the examination region selected in step S101 of image file generation processing. The control section 15 performs pop up display, at a predetermined position of the display section 19a, of the examination point selection image SI including examination point marks SM located at positions corresponding to the respective examination points set for the examination region.

In step S201, when touch to the touch panel 19b was detected at a position corresponding to the left and right switching button B5 shown in FIGS. 5 and 6, the control section 15 switches the examination point selection image SI according to left hand (right hand) to the examination point selection image SI according to right hand (left hand) to be displayed. When touch to the touch panel 19b was detected at a position corresponding to the front back switching button B6, the control section 15 switches the examination point selection image SI according to the palm side (back side) to the examination point selection image SI according to the back side (palm side) to be displayed.

The control section 15 determines whether an examination point mark SM was selected by a touch operation to the touch panel 19b at a position corresponding to one of the plurality of examination point marks SM in the examination point selection image SI (step S202).

If it is determined that the examination point mark SM was selected (step S202; YES), the control section 15 displays the body mark BM including the region figure BIc and the probe mark PM so as to be superposed on the ultrasound image UD (step S203) That is, when an operation signal corresponding to the input operation to the touch panel 19b selecting the examination point mark SM is output from the touch panel 19b in step S202, the control section 15 displays the region figure BIc and displays the probe mark PM at the position corresponding to the examination point according to the selected examination mark SM in the region figure BIc. The control section 15 displays the probe mark PM so that the direction of probe mark PM is consistent with the probe direction which is set for the examination point and stored in the HDD 152.

In step S203, the control section 15 generates composite image data combining the ultrasound image UD with the body mark BM and having a configuration as described above, stores the composite image data in the storage section 17, and controls the display section 19a to perform display based on the composite image data.

When the processing of step S203 is finished, the control section 15 determines whether a mode end operation instructing the end of body mark input mode was performed to the operation display section 19 (step S204). The control section 15 determines that the mode end operation was performed when touch to the touch panel 19b at a position corresponding to the close button B7 shown in FIGS. 5 and 6 is detected. If it is determined that the mode end operation was performed (step S204; YES), the control section 15 closes the examination point selection image SI to end the body mark display processing.

If it is not determined that the mode end operation was performed (step S204; NO), the control section 15 determines whether a body mark correction operation instructing correction of body mark BM was performed (step S205). The control section 15 determines that the body mark correction operation was performed when touch to the touch panel 19b was detected again at the position corresponding to the examination point mark SM selected in step S202. If it is determined that the body mark correction operation was performed (step S205; YES), the control section 15 deletes the body mark BM, and proceeds to the processing of step S201. If it is not determined that the body mark correction operation was performed (step S205; NO), the processing proceeds to step S204.

In step S202, if it is not determined that the examination point mark SM was selected (step S202; NO), the control section 15 determines whether the mode end operation was performed (step S206). If it is not determined that the mode end operation was performed (step S206; NO), the processing proceeds to step S202. If it is determined that the mode end operation was performed (step S206; YES), the control section 15 closes the examination point selection image SI to end the body mark display processing.

As described above, the ultrasound diagnostic apparatus 1 according to the embodiment includes: an ultrasound probe 20 which transmits ultrasound waves to a predetermined subject and receives the reflected waves of the transmitted ultrasound waves; an HDD 152 which stores examination points and directions to locate the ultrasound probe 20 so as to be associated with each other, the examination points being set for a region an examination target of the subject, and the directions to locate the ultrasound probe 20 being set in advance according to the respective examination points; a display section 19a; a control section 15 as a display control section which controls display by the display section 19a; and a touch panel 19b of an operation display section 19 which receives an input operation from outside. The touch panel 19b receives an input operation specifying the examination point, and the control section 15 (display controller) controls the display section 19a to display a body mark BM including a region figure BIc which shows the shape of the region and a probe mark PM which is located at the position corresponding to the examination point specified by the input operation and shows the direction to locate the ultrasound probe 20 set for the examination point. Thus, the operator does not need to perform the input operation for setting the direction of probe mark PM, and can display the body mark BM including the probe mark PM showing the appropriate position and direction easily by an intuitive input operation. Since the input operation for setting the direction of probe mark PM can be omitted, it is possible to display the body mark BM in a shorter time and shorten the time required for diagnosis using the ultrasound diagnostic apparatus 1. It is also possible to reduce the display area in the display section 19a required for input processing for displaying body mark BM compared to the input method of displaying a plurality of body mark candidates which have different positions and directions of probe mark and selecting a specific body mark from among the candidates, for example. Thus, the operator can perform another display such as display of the ultrasound image UD on the display section 19a while performing input processing for displaying the body mark BM, thus improving the operator convenience.

The control section 15 (display controller) controls the display section 19a to display a plurality of examination point marks SM showing examination points according to the region, and the touch panel 19b receives, as an input operation specifying an examination point, the input operation selecting one of the plurality of examination point marks SM displayed on the display section 19a. Thus, it is possible to easily display the body mark BM including the probe mark PM showing the appropriate position and direction by the intuitive input operation selecting the examination point mark SM. Since the position candidate of probe mark PM in the body mark BM is shown by the examination point marks SM, it is possible to suppress the trouble of wrong setting of the position of probe mark PM by the operator.

The control section 15 (display controller) controls the display section 19a to display a plurality of examination point marks SM so as to be located at positions respectively corresponding to the examination points according to the plurality of examination point marks SM in the region figure BIa showing the shape of the region. Thus, it is possible to display the body mark BM including the probe mark PM showing the appropriate position and direction by the intuitive input operation selecting the examination point mark SM located at the examination point in the region figure BIa.

The ultrasound diagnostic apparatus 1 includes an image processing section 16 which generates an ultrasound image UD based on the reflected waves received by the ultrasound probe 20, and the control section 15 (display controller) controls the display section 19a to display the ultrasound image UD together with the plurality of examination point marks SM. Thus, it is possible to make the operator perform input operation for selecting the examination point mark SM while visually confirming the ultrasound image UD.

The control section 15 (display controller) controls the display section 19a to display the examination point selection image SI including the plurality of examination point marks SM so as to overlap a predetermined position of the ultrasound image UD. Thus, it is possible to make the operator perform input operation selecting the examination point mark SM while visually confirming the ultrasound image UD. It is also possible to make the operator intuitively recognize that the body mark display processing of selecting the examination point mark SM and displaying the body mark BM is being performed.

The operation display section 19 includes a touch panel 19b located so as to be superposed on the display screen of display section 19a, and receives the input operation by detecting touch to the surface of the touch panel 19b. Thus, it is possible to make the operator select the examination point mark SM by an intuitive input operation of touching the position corresponding to the examination point mark SM displayed on the display section 19a in the touch panel 19b.

### Modification Example

Next, a modification example of the embodiment will be described.

In the modification example, the scan operation is started after the body mark display processing is performed in the image file generation processing. Since the other respects are similar to those of the above embodiment, hereinafter, the explanation will be made mainly for the difference between the modification example and the embodiment.

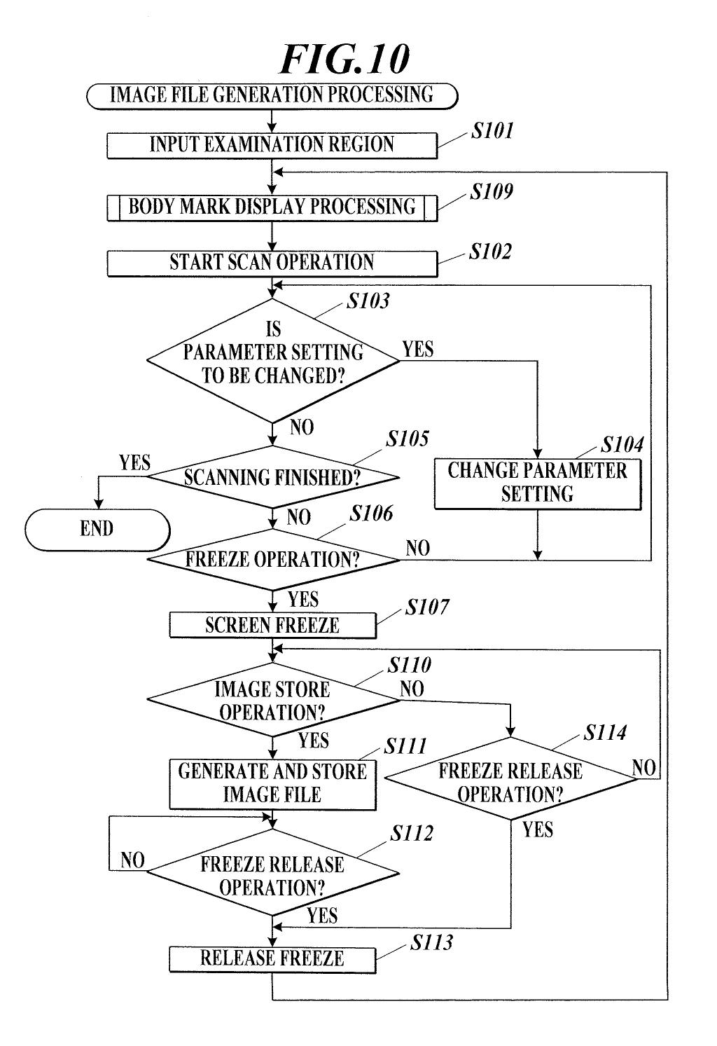

FIG. 10 is a flow chart showing a control procedure of image file generation processing according to the modification example. The flow chart is obtained by deleting step S108, changing the timing to perform step S109 to the timing after end of the step S101 and changing the processing after the end of step S113 to step S109 in the flow chart of FIG. 8. Since the other respects are similar to those in FIG. 8 of the embodiment, the detailed description is omitted.

In the image file generation processing of the modification example, when the processing according to the examination region input of step S101 is finished, the control section 15 executes the body mark display processing of step S109. In the modification example, since the ultrasound image UD is not obtained nor displayed at the start of body mark display processing, when the body mark display processing is finished, only the body mark BM is displayed at the display area of ultrasound image UD shown in FIG. 7.

When the body mark display processing of step S109 is finished, the control section 15 proceeds to step S102 and starts the scan operation. In step S102, the control section 15 generates composite image data obtained by combining the ultrasound image UD obtained by the scan operation with the body mark BM on the ultrasound diagnostic screen D2, stores the composite image data in the storage section 17, and controls the display section 19a to display the image including the ultrasound image UD and the body mark BM on the basis of the composite image data.

In step S112, the processing proceeds to step S113 if it is determined that the freeze release operation was performed (step S112; YES), and if it is not determined that the freeze release operation was performed (step S112; NO), the control section 15 repeatedly executes the processing of step S112 until the freeze release operation is performed.

When the processing of step S113 is finished, the processing proceeds to the body mark display processing of step S109.

As described above, the ultrasound diagnostic apparatus 1 of the modification example includes an image processing section 16 which generates an ultrasound image UD based on the reflected waves received by the ultrasound probe 20, and the control section 15 (display controller) controls the display section 19a to display the ultrasound image UD together with the body mark BM after controlling the display section 19a to display the body mark BM. Thus, especially in a case of performing a typical examination for which the examination point in the examination region and the probe direction of ultrasound probe 20 are determined in advance, for example, by displaying the body mark BM on the display section 19a to show the examination point and the probe direction, it is possible to make the operator easily grasp the examination point and the direction to locate the ultrasound probe 20 in the examination, improving the operator convenience.

The present invention is not limited to the above embodiment and the modification example, and various changes can be made.

For example, in the embodiment and the modification example, the examination point mark SM in the examination point selection image SI is selected by the touch operation to the touch panel 19b; however, the present invention is not limited to this. For example, the present invention may be performed in a manner that a cursor displayed on the display section 19a is moved in accordance with the input operation to a keyboard, trackball and mouse of the operation input section 18, and the examination point mark SM indicated by the cursor is selected according to a predetermined determination operation.

The embodiment and the modification example have been described by taking an example in which each of the examination point marks SM of the examination point selection image SI is a rectangular mark; however, the shape and display manner of the examination point mark SM are not limited to those of the example. For example, the examination point mark SM may be a circle or a polygon. The examination point mark SM may include a figure indicating the probe direction set for the selected point corresponding to the examination point mark SM. The examination point mark SM may have a same shape (for example, “i” shape) as that of the probe mark PM shown in FIG. 7. The examination point mark SM is not necessarily displayed together with the region figure BIa. For example, the examination point set for the region may be displayed by text.

The embodiment and the modification example have been described by taking an example in which the examination point is specified by input operation selecting one of the plurality of examination point marks SM displayed on the display section 19a; however, the present invention is not limited to this. The examination point may be specified without displaying the examination point marks SM on the display section 19a. In this case, for example, the examination point may be specified by providing input buttons corresponding to respective examination points to the operation input section 18 and pressing the input button. The examination point may also be specified by directly inputting the examination point with the keyboard of the operation input section 18.

The embodiment and the modification example have been described by taking an example in which the probe mark PM is an “i” shaped mark including a rectangular part and a square part; however, the shape of the probe mark PM is not limited to this. The probe mark may have any shape as long as it indicates the direction to locate the ultrasound probe 20.

The embodiment and the modification example have been described by taking an example in which the body mark BM is displayed by setting the direction of probe mark PM on the basis of the probe direction which is set for the examination point corresponding to the selected examination point mark SM; however, the display processing method of probe mark PM is not limited to this. For example, image data of the body mark BM locating, at an examination point, the probe mark PM which shows the probe direction set for the examination point, may be stored for each examination point in the HDD 152 in advance so that, when an examination point mark SM is selected, the body mark BM including the probe mark PM is displayed by using the image data of body mark BM according to the examination point corresponding to the selected examination point mark SM.

The embodiment and the modification example have been described by taking an example in which a storage image file is generated by combining the ultrasound image UD in still image display with the body mark BM; however, the present invention is not limited to this. A movie storage image file may be generated from image data combining each ultrasound image UD forming the movie display with the body mark BM.

The embodiment and the modification example have been described by taking an example in which the ultrasound diagnostic apparatus 1 performs examination of hand as an examination region; however, the examination region is not limited to the hand. The present invention can be applied to examinations and medical checks of various regions such as elbow, shoulder, knee, ankle, toe, head, neck, abdomen, mammary gland, thyroid, heart and fetus to be performed by the ultrasound diagnostic apparatus 1.

The embodiment and the modification example have been described by taking an example in which the direction of probe mark PM in the body mark BM is determined by selecting an examination point mark SM in the examination point selection image SI; however, the manner of processing for displaying the body mark BM is not limited to this. For example, the body mark BM may be generated by displaying the region figure BIc in the body mark BM shown in FIG. 7 and the examination mark SM overlapping the region figure BIc in step S201 of body mark display processing, and additionally displaying the probe mark PM to the region figure BIc according to the selected examination point mark SM. In this case, while the examination point mark SM is displayed, the region figure BIc may be displayed so as to be enlarged so that the operator can select the examination mark SM easily.

According to the above embodiment, modification example and other examples, the probe mark can be displayed easily and appropriately.

Though several embodiments of the present invention have been described above, the scope of the present invention is not limited to the above embodiments, and includes the scope of inventions, which is described in the scope of claims, and the scope equivalent thereof.

The entire disclosure of Japanese Patent Application No. 2015-009059 filed on Jan. 21, 2015 including description, claims, drawings, and abstract are incorporated herein by reference in its entirety.

What is claimed is:

1. An ultrasound diagnostic apparatus, comprising:
an ultrasound probe which transmits ultrasound waves to a predetermined subject and receives reflected waves of the transmitted ultrasound waves;
a storage which stores an examination point and a direction to locate the ultrasound probe so as to be associated with each other, the examination point being set for a region that is an examination target in the subject, and the direction to locate the ultrasound probe being set for the examination point in advance;
a display;
a display controller which controls display by the display; and
an inputter which receives an input operation from outside,
wherein
the inputter receives an input operation of specifying the examination point, and
the display controller controls the display to display a region mark including a first region figure which shows a shape of the region and a probe mark which is located at a position corresponding to the examination point specified by the input operation and indicates the direction to locate the ultrasound probe that is set for the examination point.

2. The ultrasound diagnostic apparatus of claim 1, wherein
the display controller controls the display to display a plurality of examination point marks showing examination points according to the region, and
the inputter receives an input operation of selecting one of the plurality of examination point marks displayed on the display as the input operation of specifying the examination point.

3. The ultrasound diagnostic apparatus of claim 2, wherein the display controller controls the display to display the plurality of examination point marks by assigning the plurality of examination point marks to positions corresponding to the respective examination points according to the plurality of examination point marks in a second region figure which shows the shape of the region.

4. The ultrasound diagnostic apparatus of claim 2, further comprising an image generator which generates an ultrasound image based on the reflected waves received by the ultrasound probe, wherein the display controller controls the display to display the ultrasound image together with the plurality of examination point marks.

5. The ultrasound diagnostic apparatus of claim 4, wherein the display controller controls the display to display an image including the plurality of examination point marks so as to be superposed on a predetermined position of the ultrasound image.

6. The ultrasound diagnostic apparatus of claim 1, further comprising an image generator which generates an ultrasound image based on the reflected waves received by the ultrasound probe, wherein the display controller controls the display to display the ultrasound image together with the region mark after the display displays the region mark.

7. The ultrasound diagnostic apparatus of claim 1, wherein the inputter includes a touch panel which is disposed so as to be superposed on a display screen of the display, and the inputter receives the input operation by detecting touch to a surface of the touch panel.
